(19) United States
(12) Patent Application Publication
(10) Pub. No.: US 2023/0346296 A1
(43) Pub. Date: Nov. 2, 2023

(54) Monitoring of Autonomic Nervous System Activity Through Sweat Pore Activation

(71) Applicants: Jacob N. HYDE, Tucson, AZ (US); Jeffrey J. RODRIGUEZ, Tucson, AZ (US); Esther M. STERNGERG, Tucson, AZ (US); Arizona Board of Regents on Behalf of the University of Arizona, Tucson, AZ (US)

(72) Inventors: Jacob N. Hyde, Tucson, AZ (US); Jeffrey J. Rodriguez, Tucson, AZ (US); Esther M. Sternberg, Tucson, AZ (US); Johnny R. Runyon, II, Tucson, AZ (US); Mohammad S. Majdi, Tucson, AZ (US)

(21) Appl. No.: 18/026,771

(22) PCT Filed: Sep. 17, 2021
(86) PCT No.: PCT/US2021/071502

§ 371 Date: Mar. 16, 2023

Related U.S. Application Data
(60) Provisional application No. 63/080,308, filed on Sep. 18, 2020.

(51) Int. Cl. A61B 5/00; A61B 5/01; A61B 5/16

(52) U.S. Cl. CPC A61B 5/4035; A61B 5/015; A61B 5/4266; A61B 5/165

(57) ABSTRACT

Various examples are provided related to monitoring of nervous system activity. In one example, a method includes obtaining at least one image of a portion of skin of a subject wherein the subject is not in contact with an imaging system and processing the at least one image to determine at least one characteristic of activated sweat pores in the at least one image. The image(s) can include at least one thermal image. The image(s) can be processed to generate at least one enhanced image for the SPA determination. In another example, a system includes an infrared camera that can capture at least one thermal image of a portion of skin of a subject with the system not in contact with the subject and image processing circuitry that can process the at least one thermal image to determine at least one characteristic of activated sweat pores in the thermal image(s).

### CROSS REFERENCE TO RELATED APPLICATIONS

This application claims priority to, and the benefit of, co-pending U.S. provisional application entitled “Monitoring of Autonomic Nervous System Activity Through Sweat Pore Activation” having Ser. No. 63/080,308, filed Sep. 18, 2020, which is hereby incorporated by reference in its entirety.

### STATEMENT REGARDING FEDERALLY SPONSORED RESEARCH OR DEVELOPMENT

This invention was made with government support under Grant Number FA8650-16-C-6762 awarded by Air Force Material Command. The government has certain rights in this invention.

### BACKGROUND

Human skin and sweat have been reported to contain many molecular biomarkers that can provide information on inflammation, diet, arousal, anxiety, pain, hygiene and more. Most research to date has focused on the secretion of distinct molecular/electrolyte biomarkers in sweat. Eccrine and apocrine sweat glands comprise the two major types of sweat glands in the human body. Eccrine glands make up 90% of all sweat glands and are found all over the human body with a density of 20-500 glands per cm². Apocrine glands are the other major type of sweat gland and are primarily innervated by beta adrenergic neurons and activated via catecholamine neurotransmitters (e.g., epinephrine and norepinephrine).

### SUMMARY

Aspects of the present disclosure are related to monitoring of nervous system activity. The measurement of sweat pore activation (SPA) can be used as a viable biomarker for autonomic nervous system activity. In one aspect, among others, a method for determining sweat pore activation comprises obtaining at least one image of a portion of skin of a subject by an imaging system wherein the subject is not in contact with the imaging system; and processing the at least one image to determine at least one characteristic of activated sweat pores in the at least one image. In one or more aspects, the at least one image can comprise at least one thermal image. Processing the at least one image can comprise generating at least one enhanced thermal image, wherein the at least one characteristic can be determined based upon the at least one enhanced thermal image. In some aspect, the portion of skin of the subject can comprise iodine and starch on the portion of skin. Processing the at least one image can comprise generating at least one enhanced image, wherein the at least one characteristic can be determined based upon the at least one enhanced image. In various aspects, the method can further comprise identifying an autonomic nervous system (ANS) status based upon the at least one characteristic. The at least one characteristic can comprise a number of pores activated in the at least one enhanced thermal image. The ANS status can be identified based upon the number of activated pores in the at least one enhanced thermal image. The ANS status can be identified based upon patterns of the activated pores. Outliers can be removed from the at least one thermal image based upon pixel size.

In various aspects, identifying the ANS status can comprise determining a stress level of the subject. Identifying the ANS status can comprise determining a source of the ANS status. The source of the ANS status can comprise a physical stressor, a physiological stressor, or an emotional stressor. The at least one image can comprise at least one video frame, captured by an imaging device at a rate of 10 frames per second (fps) or greater. Processing the at least one image can comprise filtering the at least one image. In some aspects, the at least one image can be obtained with an infrared camera of the thermal imaging system. The at least one characteristic can comprise one or more of location, size, shape, area or distribution of the pores.

In another aspect, a system for determining sweat pore activation comprises an infrared camera configured to capture at least one thermal image of a portion of skin of a subject wherein the subject is not in contact with the system; and image processing circuitry configured to process the at least one thermal image to determine at least one characteristic of activated sweat pores in the at least one thermal image. In one or more aspects, the image processing circuitry can be configured to generate an enhanced image from the at least one thermal image. The image processing circuitry can comprise a processor and a memory communicably coupled to the processor. The memory can store processor instructions that, when executed by the processor, can cause the processing circuitry to determine, by an artificial neural network, at least one characteristic of the activated sweat pores based on processing the at least one thermal image. An autonomic nervous system (ANS) status can be identified based upon the at least one characteristic. The ANS status can be identified based upon a number of activated sweat pores in the at least one thermal image. The ANS status can be identified based upon patterns of the activated sweat pores. In various aspects, the at least one characteristic can comprise one or more of location, size, shape, area or distribution of the pores.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates an example of a SPA monitoring system in accordance with various embodiments of the present disclosure.

FIG. 2 is a schematic diagram illustrating an example of image processing circuitry for implementing SPA analysis, in accordance with various embodiments of the present disclosure.

FIG. 3A illustrates an example of a thermal image processing framework for pore monitoring or detection, in accordance with various embodiments of the present disclosure.

FIG. 3B illustrates steps of a designed filter example for the enhancement in FIG. 3A, in accordance with various embodiments of the present disclosure.

FIG. 3C illustrates examples of different enhancement and denoising filters that can be used for the enhancement in FIG. 3A, in accordance with various embodiments of the present disclosure.

FIG. 4 illustrates an example of an image processing system using an artificial neural network (ANN), in accordance with various embodiments of the present disclosure.

FIG. 5 shows an example of an image of a starch-iodine skin patch and the corresponding detected sweat pore activation, in accordance with various embodiments of the present disclosure.

FIGS. 6A-6C illustrate examples of detected sweat pore information based upon raw thermal imaging data sets collected for testing, along with pupil dilation information, in accordance with various embodiments of the present disclosure.

FIGS. 7A and 7B illustrate a comparison of the pupil dilation and pore sweat area for the mental, physical and off-task conditions, in accordance with various embodiments of the present disclosure.

FIG. 8 includes images illustrating examples of original and enhanced thermal images and the detected pores, in accordance with various embodiments of the present disclosure.

FIG. 9 illustrates the change in SPA over time determined from the thermal images and sweat pore histograms for three example time points, in accordance with various embodiments of the present disclosure.

### DETAILED DESCRIPTION

Disclosed herein are various examples related to monitoring of nervous system activity. The measurement of sweat pore activation (SPA) can be used as a viable biomarker for autonomic nervous system activity. Reference will now be made in detail to the description of the embodiments as illustrated in the drawings, wherein like reference numbers indicate like parts throughout the several views.

Physical and mental effort is known to modulate autonomic arousal which can drive the activation of sweat pores. Measurements of SPA, e.g., sweat secretion and pulsation, and molecular biomarkers can provide insight into the physiology and functioning of the brain and multiple processes in the human body. Forward-looking infrared thermography can capture SPA in real time in response to both physical and mental performance tasks and can be incorporated with measurement strategies to provide tools which can be used to gain insight into the role of sweat(ing) in the modulation of the autonomic nervous system (ANS). The ANS regulates the functioning of the body's internal organs and is composed of two branches, the sympathetic nervous system (SNS) and the parasympathetic nervous system (PNS).

The SNS is responsible for the fight/flight/freeze reaction(s) in response to stressful stimuli, while the PNS is the rest/digest system responsible for the body's homeostasis and maintenance of low heart rate and muscle rest. Currently, measurement and monitoring of the ANS utilizes equipment and devices that are cumbersome, invasive, movement-restricting, and could create signals that contain unwanted signal noise or be subject to signals intelligence interception. Most commonly, ANS measurement is done via heart rate variability (HRV), skin conductance response (SCR), pupillometry/eye tracking, electroencephalography (EEG) and blood pressure. Recent advances in technology have allowed for wearable monitoring of some aspects of physiology such as HRV and gait which can be integrated into precision medicine strategies, but methods that utilize sweat as part of physiological analysis like SCR still need wiring to a device and subsequent linking to hardware.

Eccrine glands release sweat under different conditions and serve several physiological functions: (a) sympathetic nervous system activation stimulated by the fight, flight, or freeze response, (b) thermoregulation triggered by temperature changes, and (c) physical response during emotional stress. Eccrine glands are predominantly innervated via cholinergic neurons in the sympathetic nervous system (SNS), which when activated, release acetylcholine in contrast to other SNS neurons that release epinephrine/norepinephrine. This in turn stimulates contraction of myoepithelial cells at the eccrine gland, causing release of eccrine sweat. Interestingly, acetylcholine is also the predominant neurotransmitter in the parasympathetic nervous system (PNS); eccrine glands are the exception given acetylcholine's role in eccrine sweat release via sympathetic stimulation.

A stressful stimulus can cause catecholamines to stimulate myoepithelial cells at the base of the gland to contract and release apocrine sweat; catecholamines also play a role in eccrine sweat activation during emotional stress especially at plantar and palmer sites. Due to the predominance of eccrine sweat glands, their response to both temperature and stress, and their innervation by both cholinergic and nearby adrenergic neurons, these glands are ideal for further exploration of regulation of the human sweat response. The activation of these eccrine sweat pores and measurement of their secretion and pulsation patterns with respect to number, area and duration of activation, may also provide insight into modulation responses of the ANS, SNS and/or PNS to external stimuli.

Imaging of a few eccrine sweat pores on a small number of subjects can be performed through optical coherence tomography (OCT) which is a form of cross-sectional imaging at the micron-scale that measures the reflection of infrared light up to 2 mm within biological tissues. OCT was used in one study to image eccrine sweat glands in three participants' fingertips while the participant was presented with a stressful mental stimulus (loud unpleasant sound). Imaging for 19 glands was performed and showed that response to mental stress was different for each of the glands, even if they were adjacent to each other. When the subject was presented with a physical stressor (hand-grip task), the eccrine glands in the opposite hand were measured, and it was found that total excess sweat was greater for the physical stressor. Taken together, these findings on this small sample suggest that a non-uniformity exists for each individual sweat gland in response to differing types of stressors.

Direct imaging and measurement of SPA can be achieved using thermography via, e.g., a high-resolution forward-looking infrared camera. The thermal imaging camera can be hidden or exposed, and the camera can be directed at various parts of a subject's body, which can be at varying distances from the camera. For example, a thermal imaging camera can be hidden in a fixed or moving location and used to detect and collect data related to sweat, stress, ANS status or response, behavior and/or sweat pore activation. A non-contact technique was used for measuring eccrine sweat gland activity using two thermal sensors, one located 11.5 cm from the volar surface of the middle and index fingers and one located 2.3 m from the forehead in 20 participants. Participants were given instructions to breathe deeply and rapidly six times over a five-minute period while respiration and SCR were measured concomitantly. Thermography detected sweat pore response at the face in 12 of 20 participants and at the fingers in 18 of 20 participants. The correlation between the thermography of eccrine glands at the fingers and SCR was found to be 0.85. Another study assessed the relationship between post-traumatic stress disorder (PTSD) and SPA during sympathetic stimulation to find clues to what conditioned threat cues may trigger PTSD symptoms. Forward-looking infrared thermography was used to monitor SPA on the fingers and face and any correlation with SCR. It was found that activation of the sweat pores on the fingertips was highly predictive of self-rated PTSD re-experiencing symptoms. These results were in line with earlier findings that postulated that sweat pore counting may be a valid measure of SNS activity.

High-resolution thermal imaging can provide direct measurement of SPA through image capture of the dynamic thermal changes on the surface of the skin at a resolution which is sufficient to capture individual sweat pore activity. Of note, thermal imaging does not actually visualize sweat pores; rather, it records the beginning of the rapid process of sweat evaporation from the surface of the pore, resulting in an image that resembles a pore. For the purposes of measuring sweat pore activity, it would be acceptable to refer to the images of the sweat evaporating off the surface of the pore as a sweat pore. Measurement of a 4 cm² area of the skin could simultaneously measure 80-2000 sweat pores at one time, assuming a density of about 20-500 pores per cm², quickly providing large data sets for relevant statistical analysis from a single collection session.

Sweat pore monitoring as a measure of human performance during tasks that require physical or mental effort is feasible. SPA measurements were obtained from a high-resolution thermal imaging camera and were compared against pupillometry, one of the most validated methods of psychophysiological monitoring. Sweat pore activity can increase with both types of effort (mental/physical), and this change can be rapid—on a timescale of tens of seconds. Sweat pore activity can provide a sensitive index of autonomic arousal and may correlate with other measures of arousal such as pupil dilation.

Materials and Methods

Participants.

Participants were recruited and gave informed consent before taking part in the experiment. 18 participants took part in the study; 3 participants dropped out of the study before completion of data collection; and it was not possible to record analyzable data in 3 participants. Complete data was collected for 12 participants (4 male, 8 female; ages 18-20).

Procedure.

Study participants sat in a chair with their head placed into a chin rest 50 cm from a desk-mounted eye tracking device (e.g., EyeTribe) running at 30 Hz (30 frames per second) in order to record pupil dilation for the duration of the experiment. The left foot and ankle of the participants were uncovered, and both feet were placed on a wooden platform in a neutral position. The left foot was secured with hook and loop straps. A thermal imaging camera (e.g., a6703 by FLIR, Inc.) with a 50 mm, f/4 lens (e.g., FLIR, Inc.) was mounted 53 cm from the participants' left lateral malleolus, and captured images and other data were fed to a computing device (e.g., PC). Custom filters were developed in MATLAB to analyze the captured images of participant SPA.

Once the participants were comfortable, thermal image data began recording, eye tracking was calibrated and then began recording, and participants were instructed to follow prompts that they saw on the screen. Participants were then given thirty-five 4-mm glass beads and instructed to roll them around in their hands for 60 seconds in order to collect a baseline sample of palmar sweat. Participants then followed prompts on the screen from a program designed in MATLAB. They responded to a neurocognitive task (N-back 1&2 digits) with two difficulty conditions (easy/hard). Palmar sweat was collected in the same method after these tasks. Participants then engaged with a physical task (finger/keyboard tapping) with two difficulty conditions (easy/hard) which were randomized within task for each participant. Palmar sweat was again collected after this physical task. It should be noted that this current disclosure focuses on the collection and processing of thermal images of SPA.

Pupil Analysis Measurements.

Preprocessing. The eye tracker can use infrared imaging to compute a raw pupil dilation in arbitrary units at 30 Hz. It has been shown that this inexpensive eye tracker performs well at tracking pupil dilation. The raw pupil data for each eye was processed to remove pupil dropouts (e.g., from blinks) and outliers. Dropouts can be removed first by excluding all time points at which the pupil diameter was recorded as zero. Outliers can be removed using a moving filter with a window size of, e.g., 1000 time points (=33.3 seconds). Outliers can be defined as lying farther than 3 median absolute deviations from the median of the points inside the current window.

Averaging. The average pupil dilation can be computed as the average of the preprocessed pupil dilation signal for each epoch of the task. Epochs can be delineated by each of the tasks (e.g., easy mental task, hard mental task, easy physical task, and hard physical task), as well as for the periods between tasks (e.g., light flashing at the start; before the mental tasks; between the two mental tasks; before the physical task; between the physical tasks).

Image Analysis.

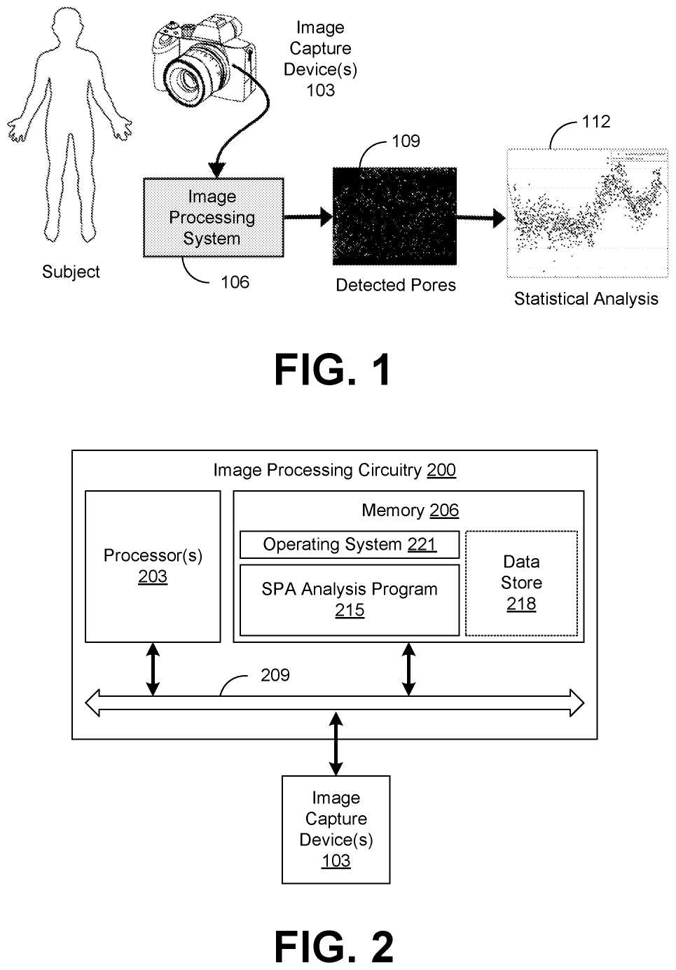

Thermography. Thermal images were recorded at 60 Hz (60 frames per second), and total image size was 640×512 pixels. FIG. 1 illustrates an example of a SPA monitoring or detection system, that includes one or more image capture device(s) 103 such as, e.g., a camera and an image processing system 106. The image capture device(s)103 can record thermal radiation in a 3-5 micron waveband. The lens can be focused manually for each participant or subject in order to maximize sharpness. In some embodiments, the image capture device(s) 103 can be fit with lenses of various focal lengths and various apertures to observe sweat pore activation from varying distances. Thermographic image data can be fed to, e.g., a processor or graphics processing unit within a computing device and a thermal analysis application (e.g., a ResearchIR Max 64-bit thermal analysis software package by FLIR Systems, Inc.). Custom-built filters can be created (e.g., in MATLAB) and imported into the thermal analysis application (e.g., the ResearchIR Max 64-bit software), which can then analyze the captured images and return information regarding detected SPA 109 (e.g., as an image with pixel values indicating detected SPA sites) and other evaluation information (e.g., in a spreadsheet or chart) from statistical analysis 112 for each participant.

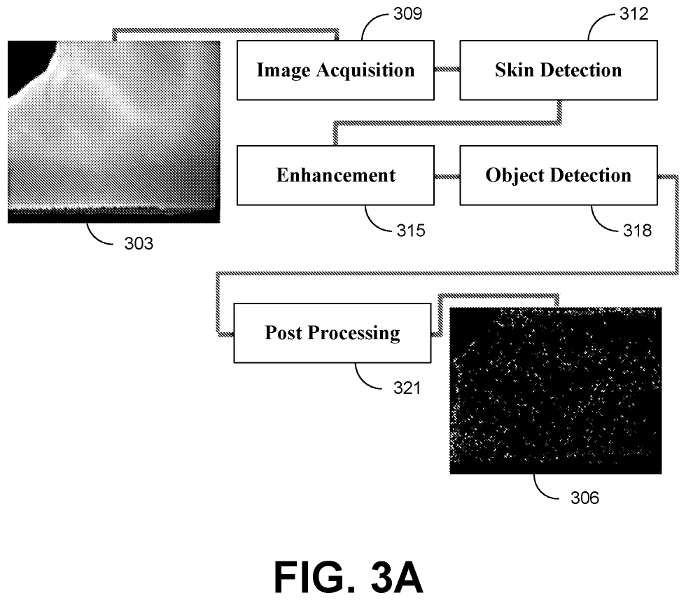

Image Processing System. Image processing circuitry comprising, e.g., a processor along with memory can be used to perform various image processing steps to determine sweat pore activation of pores in the thermal images. With reference to FIG. 2, shown is a schematic block diagram illustrating an example of image processing circuitry 200 of the image processing system 106 (or a device comprising the processing circuitry). In some embodiments, among others, the processing circuitry 200 may include a processing or computing device such as, e.g., a smartphone, tablet, computer, etc. As illustrated in FIG. 2, the image processing circuitry 200 can include, for example, a processor 203 and a memory 206, which can be coupled to a local interface 209 comprising, for example, a data bus with an accompanying address/control bus or other bus structure as can be appreciated. To this end, the image processing circuitry 200 may comprise, for example, at least one server computer or like device, which can be utilized in a cloud-based environment. In some embodiments, the image processing circuitry can include one or more network interfaces that may comprise, for example, a wireless transmitter, a wireless transceiver, and a wireless receiver. The network interface can communicate to a remote computing device using, e.g., a Bluetooth protocol or other wireless protocol.

In some embodiments, the image processing circuitry 200 can include one or more network/communication interfaces. The network/communication interfaces may comprise, for example, a wireless transmitter, a wireless transceiver, and/or a wireless receiver. As discussed above, the network interface can communicate to a remote computing device using a Bluetooth, WiFi, or other appropriate wireless protocol. As one skilled in the art can appreciate, other wireless protocols may be used in the various embodiments of the present disclosure. In addition, the image processing circuitry 200 can be in communication with one or more image capture device(s) 103 such as, e.g., a thermal imaging device (e.g., an infrared camera) and/or an optical imaging device, any of which may be configured to capture video images. In some implementations, image capture device(s) 103 can be incorporated in a device comprising the image processing circuitry 200 and can interface through the locate interface 209.

Stored in the memory 206 can be both data and several components that are executable by the processor 203. In particular, stored in the memory 206 and executable by the processor 203 can be a SPA analysis program 215 and potentially other application program(s). Also stored in the memory 206 may be a data store 218 and other data. In addition, an operating system 221 may be stored in the memory 206 and executable by the processor 203. The memory is defined herein as including both volatile and nonvolatile memory and data storage components. Volatile components are those that do not retain data values upon loss of power. Nonvolatile components are those that retain data upon a loss of power. Thus, the memory 206 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, optical disc such as compact disc (CD) or digital versatile disc (DVD), magnetic tapes accessed via an appropriate tape drive, holographic storage, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 203 may represent multiple processors 203 and/or multiple processor cores (e.g., of a graphics processing unit), and the memory 206 may represent multiple memories 206 that operate in parallel processing circuits, respectively. In such a case, the local interface 209 may be an appropriate network that facilitates communication between any two of the multiple processors 203, between any processor 203 and any of the memories 206, or between any two of the memories 206, etc. The local interface 209 may comprise additional systems designed to coordinate this communication, including, for example, performing load balancing. The processor 203 may be of electrical or of some other available construction.

A number of software components can be stored in the memory 206 and can be executable by the processor 203. An executable program may be stored in any portion or component of the memory 206. In this respect, the term “executable” means a program file that is in a form that can ultimately be run by the processor 203. Examples of executable programs may be, for example, a compiled program that can be translated into machine code in a format that can be loaded into a random access portion of the memory 206 and run by the processor 203, source code that may be expressed in proper format such as object code that is capable of being loaded into a random access portion of the memory 206 and executed by the processor 203, or source code that may be interpreted by another executable program to generate instructions in a random access portion of the memory 206 to be executed by the processor 203, etc. In particular, stored in the memory and executable by the processor can be a sweat pore activation (SPA) analysis program, an operating system and potentially other applications. Also stored in the memory may be a data store and other data. It is understood that there may be other applications that are stored in the memory and are executable by the processor as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

Although the SPA analysis program 215 and other application program(s) or systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein. Also, any logic or application described herein, including the SPA analysis program 215, that comprises software or code can be embodied in any non-transitory computer-readable medium for use by or in connection with an instruction execution system such as, for example, a processor 203 in a computer system or other processing circuitry, device or system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random-access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Pore Detection. A fast, robust and accurate image enhancement step can be adapted into the image processing system 106 (FIG. 1) to improve performance. Although many existing image enhancement techniques can improve the overall image quality, many of them lack the robustness to changes in image acquisition conditions and thus require multiple parameter tuning for different subjects and experiments. Here, a generalizable, accurate and fast technique was developed that can be implemented in real time (e.g., defined as a frame rate of 10 fps or faster), and thus, many of the more sophisticated techniques may be avoided due to their weaker generalizability or slower speed. The captured images (or frames) can be normalized using a min-max normalization with the final intensity values ranging from 0 (coldest) to 1 (warmest).

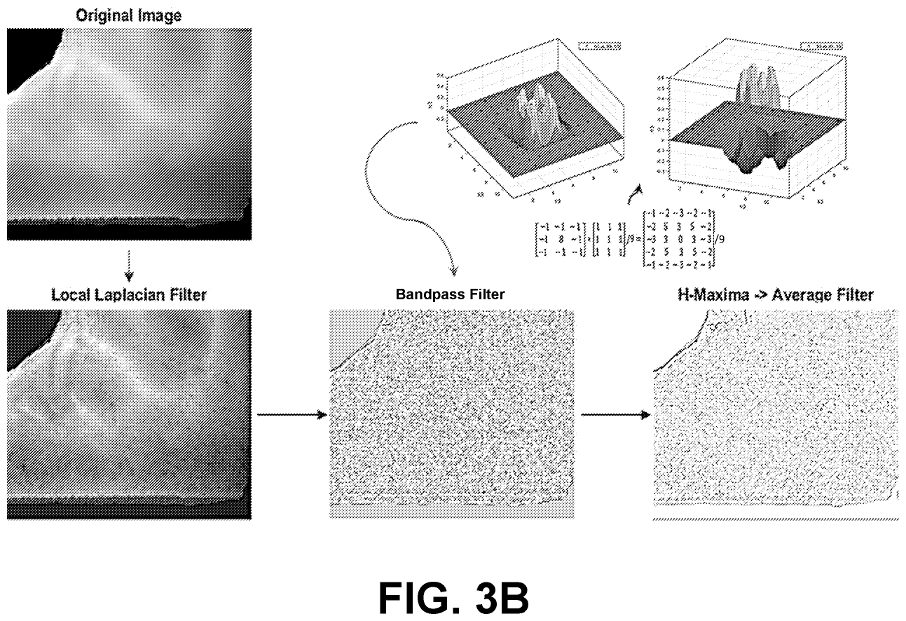

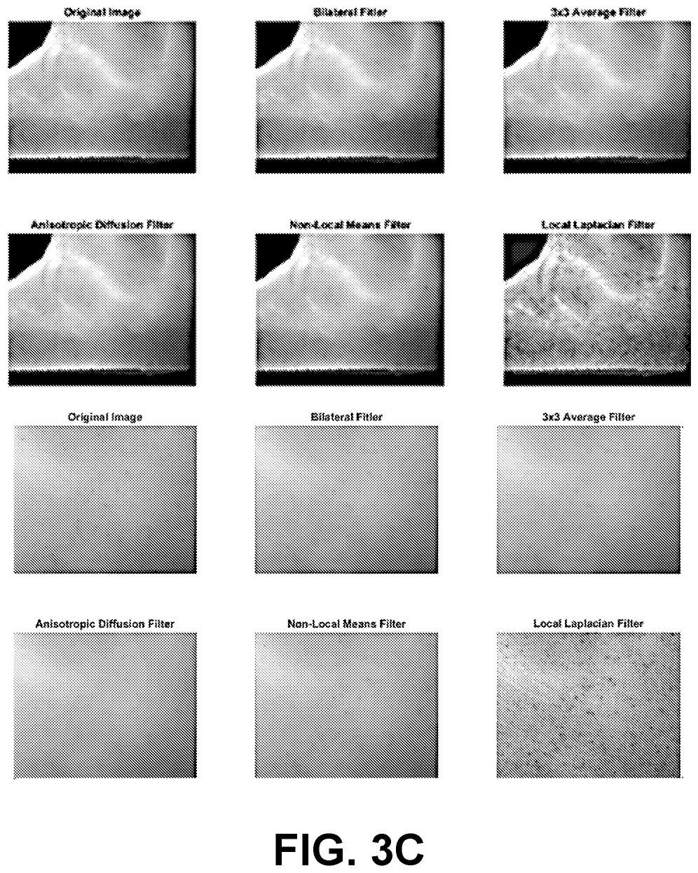

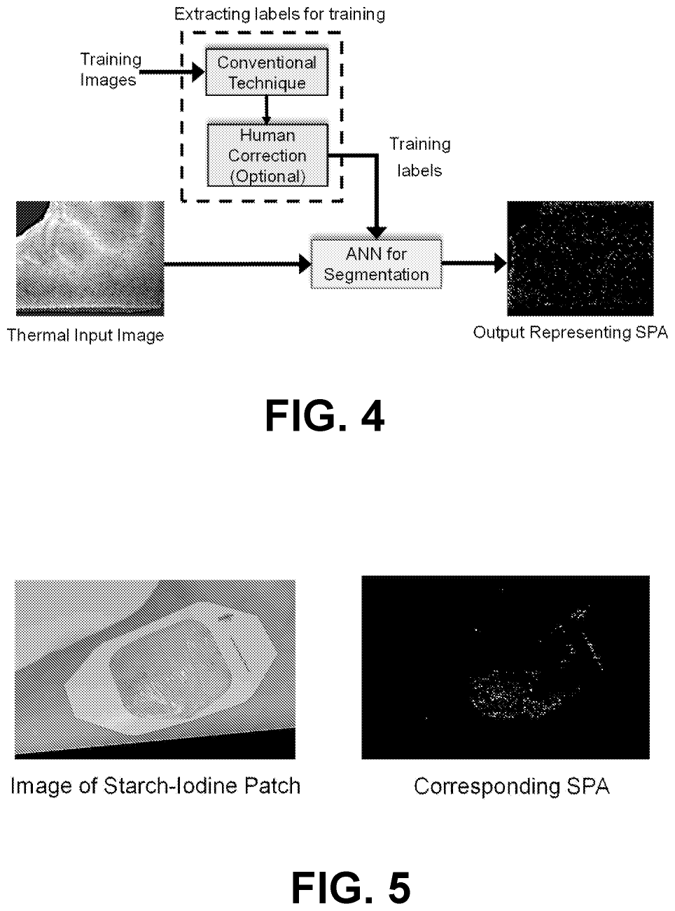

Referring to FIG. 3A, shown is an example of a pore detection image processing algorithm that can be implemented as part of the SPA analysis program 215 (FIG. 2). Original image 303 can be a sample frame from a thermal imaging video of the left ankle of a participant is shown on the left in FIG. 3A. The image 303 illustrates the appearance of the skin of a subject when the image is rendered and displayed on the screen using, e.g., the Research Max IR 64-bit software package. The dark dots represent localized areas on the surface of the skin which are cooler due to evaporation of sweat. These spots are dynamic and flash on and off during the course of the performed tasks. Image acquisition of the skin surface can be carried out at a defined rate (fps) using video and/or thermal imaging (or other appropriate imaging) at 309. Foreground detection of the captured images can be implemented at 312 to remove sweat pore candidates outside the foreground area. Enhancement of the images can then be applied at 315 (e.g., for contrast enhancement, noise reduction, sharpening, deblurring, or resolution enhancement). At 318, object detection can be performed to detect the pores. Post-processing can then be carried out at 321 (e.g., to select the appropriate objects that are representative of sweat pores based upon defined spatial characteristics of pores). A SPA image 306 indicates SPA sites (white) generated as an output of the image processing algorithm when applied to the image 303, as shown on the right. A larger number of SPA sites may be physically present, but not all are detected, due to imaging constraints (e.g., related to contrast, signal-to-noise ratio, focus, ambient lighting, or heat flow in the environment).

FIG. 3B illustrates an example of enhancement 315 of an original image of sweat pores by local Laplacian filtering, bandpass filtering (e.g., 3×3 Laplacian combined with 3×3 local averaging), h-maxima transform, and local averaging. FIG. 3C illustrates the effects of alternative, example filters for enhancement 315 of the images. In FIG. 3C, the bottom six images show zoomed sub-images of the corresponding top six images. Alternatively, other types of image enhancement may be used—e.g., contrast enhancement, noise reduction, sharpening, deblurring, or resolution enhancement, The choice of enhancement technique may involve tradeoffs between generalizability, accuracy, and speed.

One technique for enhancement 315 is to enhance the contrast while preventing oversaturation in homogeneous areas by using contrast limited adaptive histogram equalization (CLAHE). For example, each image (e.g., 640×512 pixels) can be divided into 50 rows and 50 columns of blocks, each having size of, e.g., 13×11 pixels. A histogram can be calculated for each of these blocks. The number of bins can be selected in a trade-off between high dynamic range (more bins) and faster processing speed (fewer bins). Finally, a median filter (e.g., a 3×3 median filter) can be performed on each image, where each pixel output contains the median value in a neighborhood (e.g., a 3×3 neighborhood) around the corresponding input pixel.

Object detection 318 can be performed as follows: First, the enhanced image can be binarized using either local or global thresholding. Second, the binarized input can be classified into objects by locating all white pixels that are connected in an 8-connectivity neighborhood. For example, to segment the enhanced pores, adaptive thresholding (e.g., an adaptive Otsu thresholding) can be used. The enhanced images can be binarized using thresholds based on the local mean intensity in their neighborhood. The binarized images can be segmented or partitioned into objects by locating all the connected regions of thresholded pixels—e.g., using connected components labeling with 8-connectivity.

Post-processing 321 can be applied to select the appropriate objects that are representative of sweat pores based upon defined spatial characteristics of pores. This can include filtering the objects based upon their size, shape, and location in the image with respect to the foreground area. For example, a series of morphological filters can be performed to select the more likely candidates that could represent the actual sweat pores. A closing operation with a circular disk structure can be performed to join objects that may have been separated due to noise. Detected objects smaller than 2 pixels or larger than 35 pixels can be removed. All detected candidates outside the foreground area selected by the user can be removed. Finally, to eliminate outliers, all frames with either the number of detected pores or their accumulated pore area more than three standard deviations from the median (over all frames corresponding to that subject) can be removed.

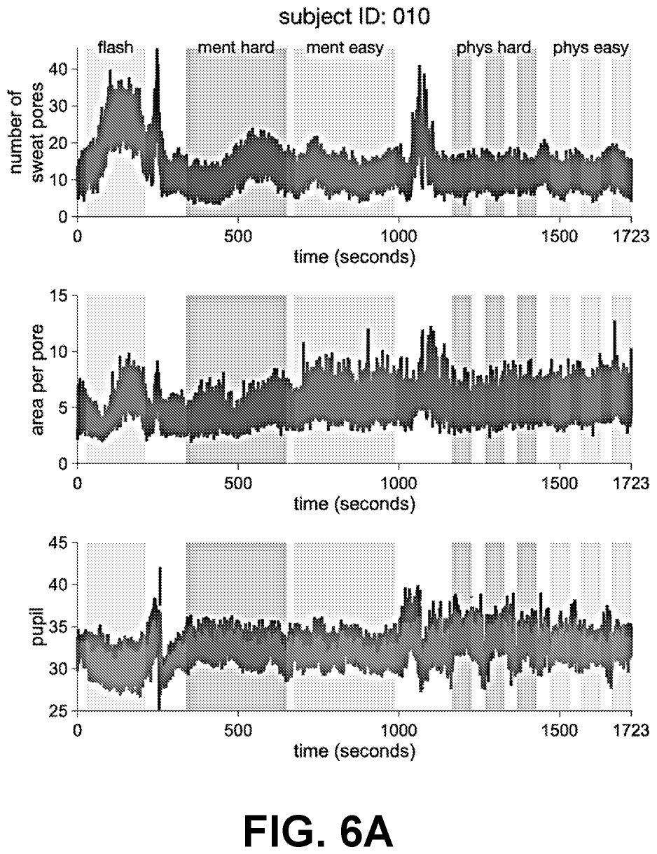

Referring to FIG. 4, shown is an example of image processing using an artificial neural network (ANN). In various implementations, an ANN can be used—e.g., a convolutional neural network (CNN) or deep neural network (DNN). The parameters of the ANN can be trained on a set of training images, where sweat pore locations are indicated, e.g. by labels. For example, a machine learning architecture can be used, along with an appropriate training algorithm and/or cost function. For example, a Densenet121 architecture can be trained using training data such as manually labeled data and/or predictions obtained from conventional image segmentation algorithms such as, e.g., adaptive Otsu thresholding, or from the techniques described herein. The training data may also include information derived from images of a starch-iodine skin patch. The ANN can go one step further and estimate the uncertainty in detected pores for each image, and thus improve the overall confidence of the results. After training the parameters of the ANN, a thermal image can be input to the ANN, which then computes an output representing the determined SPA as illustrated in FIG. 4.

Alternatively, the implementation of the ANN may be accomplished using object detection techniques. For example, one technique is to use a high-speed, single-stage, object detection system, such as YOLOv3 (which includes residual blocks, skip connections, and multiscale detection) or RetinaNet. An alternative technique with slower speed, but better localization and recognition accuracy, is to employ a two-stage object detection technique such as Fast RCNN or Faster RCNN in conjunction with an FPN, ResNet, or ResNeXT backbone. Single-stage detectors determine SPA sites directly from input images, eliminating the need for a region proposal phase, making them extremely efficient and suitable for real-time devices. Two-stage detectors can be separated into two stages by an RoI (region of interest) pooling layer. For example, in Faster R-CNN, a first stage, called RPN (region proposal network), generates potential SPA bounding boxes. A second stage involves extracting features from each potential SPA bounding box using the RoI Pool (RoI pooling) method for the classification and bounding-box regression tasks that follow. The choice between a single-stage or two-stage detector, or other type of ANN object detector, can be made according to the desired balance of accuracy, processing speed, and computational availability. The ANN object detector may use additional techniques such as divided grid cells, multiscale feature maps, and novel loss functions for improved performance.

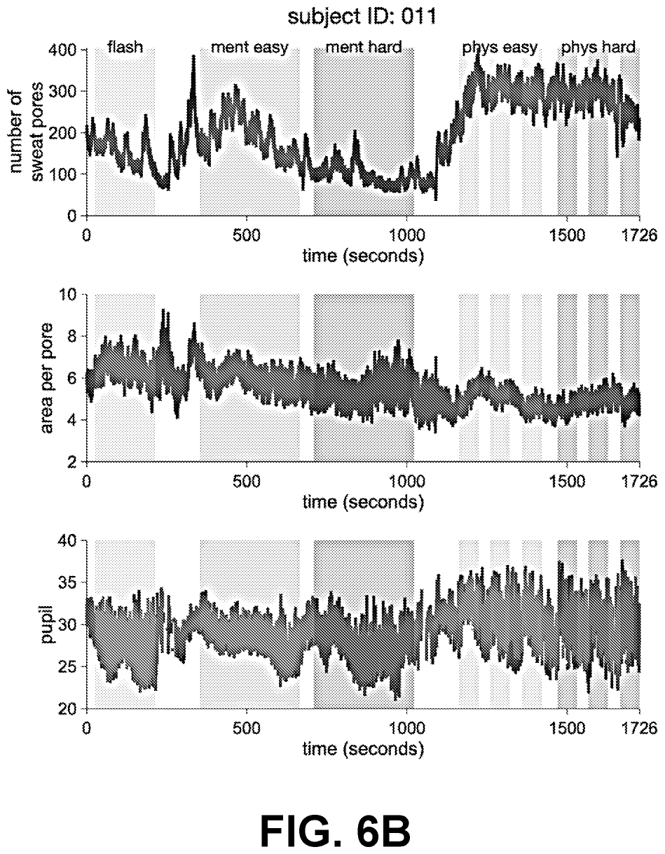

Another alternative technique to determine SPA involves the use of a starch-iodine skin patch as illustrated in FIG. 5. A starch-iodine skin patch is a portion of the skin of a subject where starch and iodine have been applied, as shown in the left image of FIG. 5. For example, one technique is to apply an iodine solution to a portion of the skin, apply a thin layer of starch to the sticky side of a transparent film dressing (e.g., Tegaderm patch by 3M), and affix the film dressing to the portion of the skin. A chemical reaction with sweat will cause various spots within the starch-iodine skin patch to turn a dark color (e.g., purple or blue), indicating that sweat glands have been activated. An optical imaging camera can be used to capture at least one optical image or video frame of a starch-iodine skin patch, with or without a film dressing being present. In some embodiments, the image capture device(s) 103 can be fit with lenses of various focal lengths and various apertures to observe sweat pore activation from varying distances. Images can be recorded in the visible band at, e.g., 60 Hz (60 frames per second), and with an image size of, e.g., 640×512 pixels. Similar to the processing of thermal images of sweat pores, optical images of a starch-iodine skin patch may be processed using image processing, image analysis, and/or machine learning techniques in order to determine SPA, resulting in output information regarding SPA, as shown in the right image of FIG. 5. This may be implemented by the SPA analysis program. For example, the SPA information determined by applying the image processing system disclosed herein to optical images of a starch-iodine skin patch may be used as training data for an ANN, which may be in addition to other training data. This processing of a starch-iodine skin patch to determine SPA may also be used to validate or improve the accuracy of the results of the analysis of thermal images of the skin of a subject.

Statistical Methods for Analyzing Pupil and Sweat Data.

The main effects of task type can be assessed (e.g., physical vs. mental vs. off-task), task order (e.g., hard first vs. easy first), and task difficulty (e.g., hard vs. easy) using repeated measures analysis of variance (ANOVA) with task type and task difficulty as within-subjects factors; task order was a between-subjects factor. To assess whether task difficulty had a separate effect on pupil and sweat response in the mental and physical tasks, a repeated measures ANOVA was performed on just the data from the mental or physical tasks with task order (e.g., hard first vs. easy first), as a between-subjects factor, and task difficulty (e.g., hard vs. easy), as a within-subjects factor. In addition, an interaction term to test for the possibility that the task order interfered with the response to task difficulty was included.

Results

Sweat pores present as pulsing dark spots in thermal images when a grayscale palette is utilized; the temperature scale shows the coolest regions as black and warmest regions as white, and mid-temperature regions as various shades of gray. The image analysis algorithm described herein identifies sweat pores from captured thermal images and reports the number of pores and the average size (e.g., number of pixels) of the poresin each image.

SPA can be determined in various ways. For example, SPA can be determined by calculating the number of sweat pores in each image. As another example, SPA can be determined by calculating the total number of sweat pore pixels in each image.

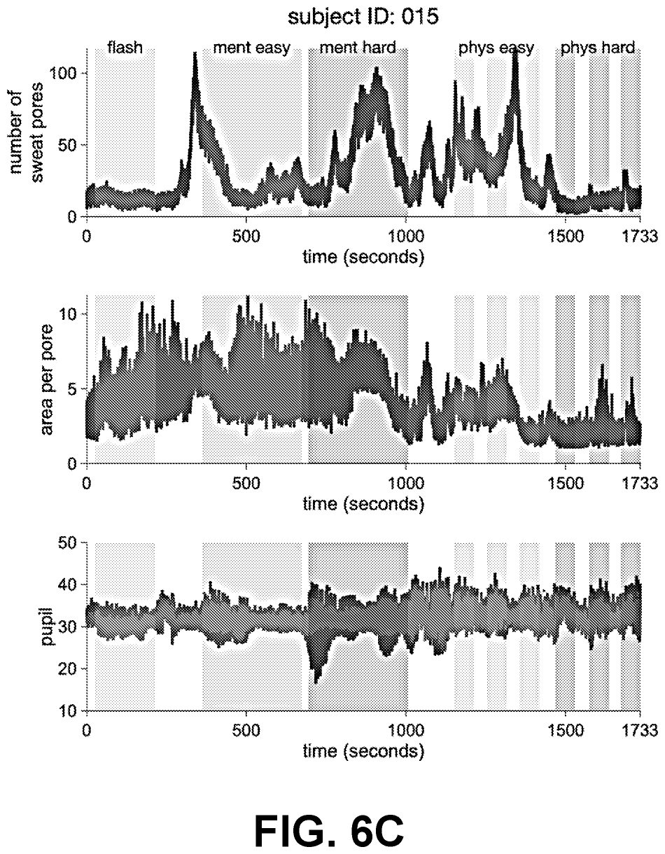

FIGS. 6A-6C show examples of raw data sets collected over the time course of the experiment for three subjects. The number of pores and area per pore (total area of all pores divided by the total number of counted pores in each image) in addition to pupil dilation were measured for each hard and easy mental and physical task. The “flash” band represents the experimental setup initiation time. The “ment hard” and “ment easy” bands are the time points for the N-back mental tasks. The “phys hard” and “phys easy” bands are the time points for the physical tasks. The order of mental task first followed by physical task was consistent for all participants while the order of hard versus easy within each task was varied between participants.

There was considerable variability in the sweat measurements across the experiment and between individuals. For individual participants, this variability appears to align with different components of the task. For example, subject 010 (FIG. 6A) showed an increased number of sweat pores during the calibration step (“flash” band), subject 011 (FIG. 6B) increased sweating during the physical task (“phys hard” and “phys easy” bands), and subject 015 (FIG. 6C) showed increased sweat response in the hard mental task (“ment hard” band). Discernment between SPA patterns which represent distinct phenotypes can be improved by collecting SPA data from a larger and more diverse cohort. However, the changes in sweat pore number are certainly real and can be readily seen in the raw images of the foot.

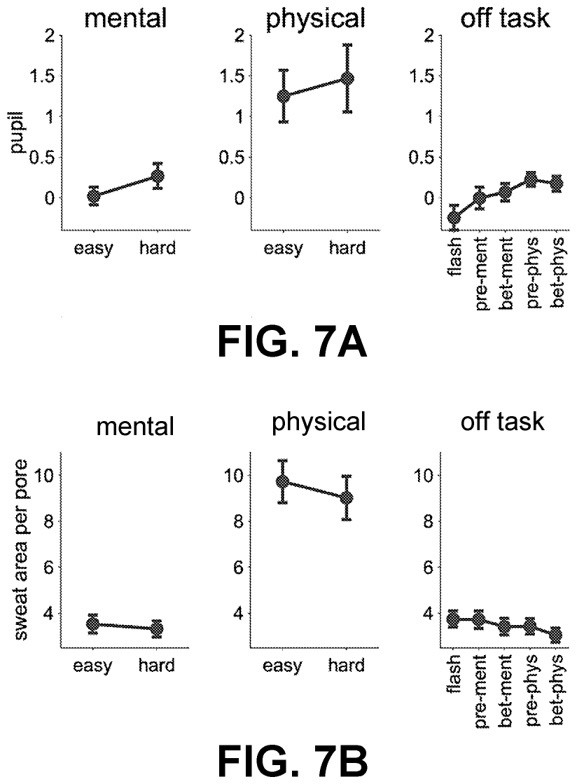

To better understand whether there were any systematic changes in sweat pore activation between conditions, data was averaged within each block. It was tested whether this aggregate sweat response changed with condition. Analysis for pupil dilation was conducted first, where the link with mental and physical effort is well established. FIG. 7A shows a comparison of the pupil dilation for the mental, physical and off-task conditions. SPA was larger for the physical task than either the mental or off-task responses (main effect of physical task on pupil: F(1, 93)=38.34, p<0.001, η²=0.22) and larger for the hard mental task than the easy mental task (main effect of task difficulty when analyzing just the mental data: F(1, 10)=17.43, p=0.002, η²=0.072). However, the numerical increase in pupil dilation between the hard and easy physical tasks was not statistically significant (main effect of task difficulty when analyzing just the physical data: F(1, 10)=0.65, p=0.44, η²=0.008).

A similar increase in sweat response in the physical task relative to the mental task or off-task periods was found when performing the same analyses on average area per pore. FIG. 7B shows a comparison of the sweat area per pore for the mental, physical and off-task conditions. The main effect of the physical task on area per pore was F(1, 93)=381.85, p<0.001, η²=0.47. In contrast to pupil responses, in which (at least numerically) pupil dilation increased in the hard conditions of the mental and physical tasks, numerically, sweat activity decreases in the hard conditions of the tasks. However, these differences are not statistically significant in either the mental (main effect of task difficulty when analyzing just the mental data: F(1, 10)=3.03, p=0.11, η²=0.0079) or physical tasks (main effect of task difficulty when analyzing just the physical data: F(1, 10)=3.72, p=0.083, η²=0.013).

Remote monitoring of sweat pore activation offers a usable, rapid and robust method to monitor the status or response of the ANS. While there are multiple existing and well-validated methods in psychophysiology to monitor the ANS, there are many industrial and occupational reasons for developing a non-invasive and no-contact method of ANS monitoring, including assessment of readiness for task performance. For example, SPA may increase with both types of effort (mental/physical) and this change can be rapid—on a timescale of tens of seconds.

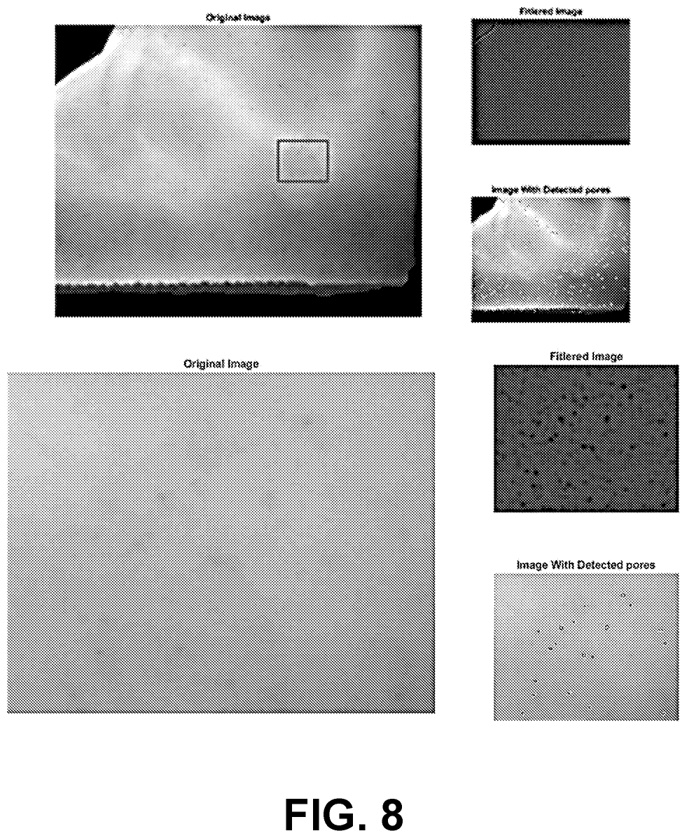

Thermal imaging can be used to capture SPA dynamics that occur on the time scale of seconds. FIG. 8 demonstrates this for subject 015 in which the original raw data shown in FIG. 6C has been smoothed using a rolling average of one-minute duration. The percent change in sweat pore number was plotted as a means of normalization.

Synchronization of thermal imaging SPA data from the thermographic camera with pupillometry data and task progression was accomplished by flashing an incandescent light bulb on and off before the start of each stage of performing mental and physical tasks. The bulb created an additional heat source and resulted in increased thermographic readings of surrounding surfaces and air through the use of software mitigation. For this, the foreground area (representing skin) in each image was cropped from the background (containing the light bulb). Oversaturation caused by the light bulb (after going through the normalization step) can be mitigated by reducing the intensity values of the light bulb area below the skin area maximum intensity value.

Different semi-supervised and unsupervised methods can be performed. For semi-supervised methods, two approaches were investigated. In the first method, a manual region of interest (ROI) can be drawn by the user, which specifies the location of the light bulb, and thus the extra brightness caused by this area of the image that would exceed the brightness in the rest of the image was capped by reducing all its pixel values to the maximum intensity within the remainder of the image area. In a second method, the foreground ROI (encompassing the skin area drawn by the user) can be used to find the maximum allowed intensity in each image, and any pixel values brighter than this can be reduced to this maximum value. A third approach that can be taken is an unsupervised method aimed at removing the dependency of the algorithm to manually drawn ROIs. A four-bin histogram of all pixel intensities in each image can be calculated in, e.g., MATLAB. The weighted average intensity of all pixels that fall within the last three bins of this histogram can be calculated and assigned as the maximum permitted brightness (Iₘₐₓ) in that image. Finally, the value of all pixels with a brightness greater than Iₘₐₓ can be reduced to Iₘₐₓ.

For three participants, the acquired video image sequences lacked the desired contrast and signal-to-noise ratio to easily distinguish the sweat pores. This became exacerbated when environmental effects (e.g., lighting effects and heat pollution) were added. As a result, data from these participants was unusable. Additionally, continuous exposure without synchronous automated camera focusing (which is beneficial in the presence of subject movement), along with changes in lighting and flow of heat in the video acquisition environment impacted the images acquired from some participants.

The observed SPA varied greatly from person to person. Activation of pores occurred under both physical and mental effort and SPA occurs on a rapid time scale as supported by the data. This heterogeneity across the population reflects meaningful individual differences in either ANS activity or behavior and may allow use of a larger and more diverse cohort and a higher differentiation between easy and hard tasks for physical and mental stimuli. However, the changes in sweat pore number are real and can be readily seen in the raw images of the foot (see FIG. 3A). The results echo previous findings which also found strong non-uniformity between sweat glands, and increased gland activity during physical tasks. Despite large variation across participants, aggregate data showed that SPA increases with physical effort compared to either mental effort or off-task SPA.

Sweat pore activity was also found to be a sensitive index of autonomic arousal and should correlate with other measures of physiological arousal such as pupil dilation. To test this, the dynamic SPA data was aggregated as shown in FIG. 7B. Pupil data showed a robust and expected trend of increased pupil size for physical over mental tasks and for hard over easy tasks. The SPA data showed increased SPA for physical compared to mental tasks in agreement with pupil data, but an opposite trend between hard vs easy tasks. The ANOVA analysis indicates that changes in SPA between easy and hard tasks was statistically insignificant. Varying the tasks themselves, randomizing the order of tasks, increasing the differential between easy and hard tasks, and measuring SPA in a larger cohort may help to better determine statistical significance in SPA measurement between easy and hard tasks. This may improve discernment between SPA patterns which represent potential distinct human stress response phenotypes.

It is also possible that sweat pores with different activation characteristics (e.g., fast or slow on/off, smaller/larger) reflect pores that are activated by different components of the ANS (sympathetic adrenergic versus cholinergic). In that case, image analysis to select pores with different characteristics may provide a window into the balance of ANS activity during different kinds of stress, and may be used to selectively discern individual differences in stress responsiveness. The image analysis can be used to detect a wide range of characteristics of sweat pores, SPA, and sweat evaporation from the skin on all parts of the body from fixed or moving distances. Characteristics can include, but are not limited to, size, shape, area, distribution, location within the image, patterns of activation/inactivation, changes dependent upon temperature variables, skin preparation differences and/or hydration status.

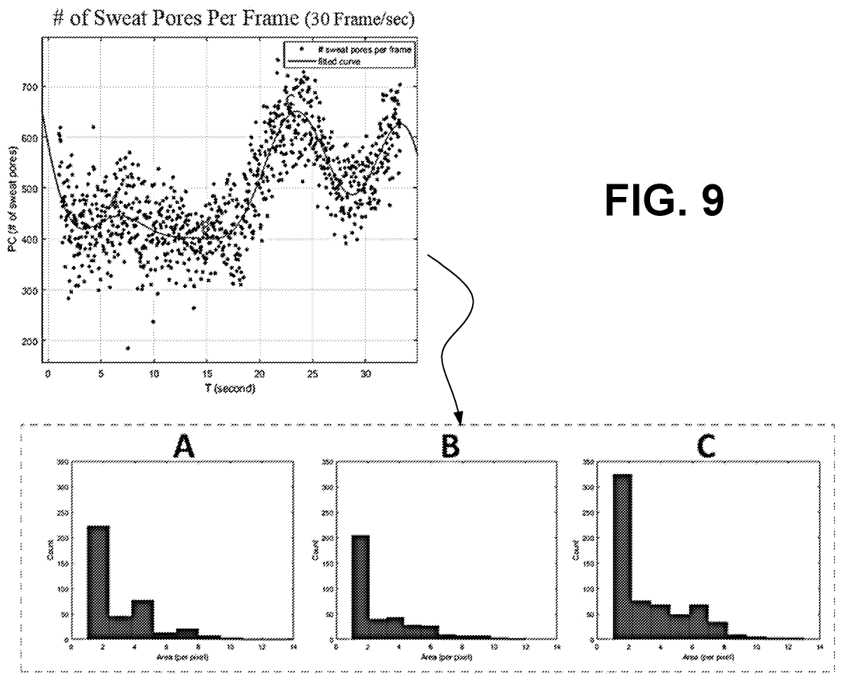

To provide a sensitive and non-invasive method to measure ANS activity, an image analysis program was developed to analyze remote observation of SPA. One of the most common applications of sweat pore detection is the discernment of liveliness in fingerprint biometry (e.g., to avoid the deception of fingerprint systems using artificial fingers). Different techniques have been employed to measure sweat pore size, density and shape in fingerprints. These existing methods assume specific pore shapes. In contrast, the disclosed analysis of hundreds of detected pores on multiple participants showed no specific spatial feature especially as a result of the appearance of multiple pores in the proximity of other pores. As seen in FIG. 9, thermography images collected in the current study show that pore structures observed on the surface of the skin can have different shapes and sizes that range from 0.2 mm² to 2 mm². This method can be extended to fingerprint detection to provide a presentation of sweat pore changes during the biometry scanning process, and thus obtain a more accurate estimate of liveliness in fingerprint biometry.

Measurement of physiological responsiveness of sweat pore activation offers a unique window into cognitive performance via the skin, the body's largest organ, whose modulation of the ANS is still not well understood. Tracking of SPA via remote thermographic imaging is a non-invasive measurement which can, in near-real time, inform the understanding of human performance during real-world industrial and occupational tasks in multiple types of settings. It has been found that SPA changes occur on the time scale of seconds under differing types of mental and physical stress tasks. Additionally, changes in SPA correlated with a standard psychophysiological measurement (pupillometry), where it was possible to differentiate between task type (mental/physical) but not task difficulty (easy/hard), likely due to small cohort size.

While there are established and validated measurement methods of ANS status, many are cumbersome, require attachment to skin, restrict movement of the wearer, are heavy and may be subject to signals intelligence interception or signal disruption. In this disclosure, a remote and non-invasive approach to measurement in order to determine whether SPA monitoring via thermography is shown to be feasible in detecting ANS changes. This methodology can be utilized to identify traits (e.g., physiological or psychological) in individuals in a wide range of occupational roles by observing sweat and/or SPA. Due to the cumbersome and overt nature of wearing multiple devices and sensors, there is a need in industrial and occupational settings for remote, non-invasive and near-real time monitoring of physiology in individuals due to health risks (e.g., SARS-CoV-2), and such monitoring may be useful for occupational task-load analysis. This type of measurement may also be useful in monitoring the performance of operators and pilots of expensive and complex equipment (e.g., machinery, naval vessels, unmanned aerial vehicles) along with individuals who would benefit from an expanded understanding of the ways in which various stressful stimuli may moderate their ANS changes (e.g., elite athletes, warfighters). This may also be used to assess and/or select individuals for occupational roles or teams that operate under stress inducing conditions. Finally, there are multiple ways this type of measurement and monitoring could be used covertly in intelligence and defense settings when collecting human intelligence data, during examinations for security clearances, identification of individuals of interest, or during assessment and selection for various types of occupational teams. For example, individuals of interest, whether known or unknown, may be identified by observing sweat and/or SPA. Observation of patterns of sweat and/or SPA known for an individual may be used to identify the individual or other individuals of interest. The observed and collected data can be applied or stored in one or more dataset(s) for use in determining the identity of individuals. The information in the dataset(s) can be used to determine, e.g., ANS status and/or stress level of an individual and may be used to distinguish between different types of stressors (e.g., physical, psychological, emotional, etc.). This methodology may also be used to identify lying, deception, or untruthfulness by observing the sweat and/or SPA. In addition, responses to different types of stimuli with emotional valance can be identified by observing the sweat and/or SPA. The dataset of information obtained from subjects can provide baseline information that can be used in such evaluations.

This disclosure has shown that remote, non-invasive measurement of ANS dysregulation is possible with thermal imaging. In some cases, remote observation and measurement may be useful within industrial and occupational settings and could be used to provide large amounts of data for analysis (e.g., over 150,000 data points for each participant were generated in the current study). This can be used for human performance monitoring within industrial and occupational settings and has potential for assessment, selection, and covert monitoring of individuals in highly sensitive occupations.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A method for determining sweat pore activation, comprising:
obtaining at least one image of a portion of skin of a subject by an imaging system wherein the subject is not in contact with the imaging system; and
processing the at least one image to determine at least one characteristic of activated sweat pores in the at least one image.

2. The method of claim 1, wherein the at least one image comprises at least one thermal image.

3. The method of claim 2, wherein processing the at least one image comprises generating at least one enhanced thermal image, wherein the at least one characteristic is determined based upon the at least one enhanced thermal image.

4. The method of claim 2, wherein processing the at least one image comprises using an artificial neural network to determine the at least one characteristic.

5. The method of claim 1, wherein the portion of skin of the subject comprises iodine and starch on the portion of skin.

6. The method of claim 5, wherein processing the at least one image comprises generating at least one enhanced image, wherein the at least one characteristic is determined based upon the at least one enhanced image.

7. The method of claim 1, further comprising:
identifying an autonomic nervous system (ANS) status based upon the at least one characteristic.

8. The method of claim 7, wherein the at least one characteristic comprises a number of pores activated in the at least one enhanced thermal image.

9. The method of claim 8, wherein the ANS status is identified based upon the number of activated pores in the at least one enhanced thermal image.

10. The method of claim 9, wherein the ANS status is identified based upon patterns of the activated pores.

11. The method of claim 10, wherein outliers are removed from the at least one thermal image based upon pixel size.

12. The method of claim 7, wherein identifying the ANS status comprises determining a stress level of the subject.

13. The method of claim 7, wherein identifying the ANS status comprises determining a source of the ANS status.

14. The method of claim 13, wherein the source of the ANS status comprises a physical stressor, a physiological stressor, or an emotional stressor.

15. The method of claim 7, wherein the at least one image comprises at least one thermal image captured by a thermal imaging device at a rate of 10 frames per second or greater.

16. The method of claim 7, wherein processing the at least one image comprises filtering the at least one image.

17. The method of claim 1, wherein the at least one image is obtained with an infrared camera.

18. The method of claim 1, wherein the at least one characteristic comprises one or more of location, size, shape, area or distribution of the activated sweat pores.

19. A system for determining sweat pore activation, comprising:
an infrared camera configured to capture at least one thermal image of a portion of skin of a subject wherein the subject is not in contact with the system; and
image processing circuitry configured to process the at least one thermal image to determine at least one characteristic of activated sweat pores in the at least one thermal image.

20. The system of claim 19, wherein the image processing circuitry is further configured to generate an enhanced image from the at least one thermal image.

21. The system of claim 19, wherein the image processing circuitry comprises:
a processor; and
a memory communicably coupled to the processor, wherein the memory stores processor instructions that, when executed by the processor, causes the processing circuitry to determine, by an artificial neural network, at least one characteristic of the activated sweat pores based on processing the at least one thermal image.

22. The system of claim 19, wherein an autonomic nervous system (ANS) status is identified based upon the at least one characteristic.

23. The system of claim 22, wherein the ANS status is identified based upon a number of activated sweat pores in the at least one thermal image.

24. The system of claim 22, wherein the ANS status is identified based upon patterns of the activated sweat pores.

25. The system of claim 19, wherein the at least one characteristic comprises one or more of location, size, shape, area or distribution of the pores.
